(19) United States
(12) Patent Application Publication
(10) Pub. No.: US 2007/0082945 A1
(43) Pub. Date: Apr. 12, 2007

(54) Discodermolide compositions

(76) Inventors: Weniel Jiang, Bridgewater, NJ (US); Saran Kumar, Edison, NJ (US); Rebecca Robertson, Westfield, NJ (US); Sui-Ming Wong, Collegeville, PA (US)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 10/578,168

(22) PCT Filed: May 11, 2004
(86) PCT No.: PCT/EP04/12557

§ 371 Date: Dec. 1, 2006

Related U.S. Application Data
(60) Provisional application No. 60/517,902, filed on Nov. 6, 2003.

(51) Int. Cl. A61K 31/366

(52) U.S. Cl. 514/460

(57) ABSTRACT

Pharmaceutical formulations comprising discodermolide in the form of an infusion concentrate, and methods of administration of discodermolide in suitable form for parenteral administration.

This invention is concerned with pharmaceutical formulations of discodermolides, and in particular pharmaceutical formulations which are administrable parenterally, e.g., intravenously (i.v.).

### BACKGROUND OF THE INVENTION

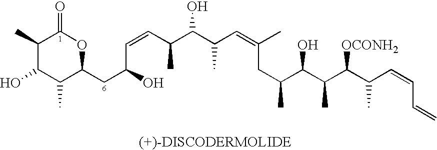

(+)-Discodermolide is a novel polyketide natural product that was isolated from extracts of the marine sponge Discodermolide dissoluta by researchers at the Harbor Branch Oceanographic Institution (HBOI). See Gunasekera et al., “Discodermolide: A New Bioactive Polyhydroxylated Lactone From the Marine Sponge Discodermia Dissolute”, J Org Chem, Vol. 55, pp. 4912-4915 (1990). Discodermolide lacks obvious structural resemblance to paclitaxel, yet it shares with paclitaxel (the active substance in the drug Taxol®) the ability to stabilize microtubules. In mechanism-based assays, discodermolide is more effective than paclitaxel. In fact, of the handful of compounds known to induce polymerization of purified tubulin, discodermolide is the most potent. However, microtubules, a major structural component in cells, are not simple equilibrium polymers of tubulin. They exist as regulated GTP-driven dynamic assemblies of heterodimers of α- and β-tubulin. Although the dynamics are relatively slow in interphase cells, upon entering mitosis, the rate of growing and shortening increases 20- to 100-fold—the average microtubule turns over half the tubulin subunits every 10 seconds. This change in rate allows the cytoskeletal microtubule network to dismantle and a bipolar spindle-shaped array of microtubules to assemble. The spindle attaches to chromosomes and moves them apart. The response to complete suppression of microtubule dynamics in cells is death. However, mitotic cells are more sensitive and the tolerance threshold appears to be cell-type specific. Molecules like paclitaxel that bind with high affinity to microtubules disrupt the dynamics process in tumor cells with lethal results even when the ratio of bound drug to tubulin is very low. Discodermolide binds to tubulin competitively with paclitaxel. Since paclitaxel has proven to be useful in treating some cancers, other compounds of the same mechanistic class may have utility against hyperproliferative disorders.

However, little has been published on formulations suitable for discodermolides. We have found that the compound has poor stability and poor solubility in aqueous solvents and aqueous-organic mixtures. The compound is practically insoluble in water. The poor solubility of these compounds makes it very difficult to form pharmaceutical formulations for parenteral administration. Concentrated solutions of the microtubule agent Taxol® which can be diluted in an aqueous medium prior to i.v. administration have been described. However, such solutions conventionally employ a surfactant, such as Cremophor® (polyethoxylated castor oil). It is well-known that surfactants, such as Cremophor® can cause allergic reactions in patients.

In addition, discodermolide contains a lactone group which can hydrolyze to form the carboxylic acid and therefore the compound is particularly labile to degradation.

Thus there is a need for commercially-acceptable pharmaceutical formulations suitable for discodermolides, e.g., pharmaceutical formulations which allow for storage, such as in a refrigerator, at 2-8° C. and/or at 25° C.

We have now surprisingly found means to improve the solubility and stability of discodermolides, and/or render them more soluble without adversely affecting their potency.

### SUMMARY OF THE INVENTION

The present invention is directed to pharmaceutical formulations comprising discodermolide which have improved solubility and stability. The invention also comprises an infusion concentrate of discodermolide which can be diluted with a diluent vehicle to produce a infusion solution prior to i.v. administration.

### DETAILED DESCRIPTION OF THE INVENTION

Discodermolides useful for treating tumor diseases and other conditions are disclosed in U.S. Pat. Nos. 5,010,099; 4,939,168; 5,840,750; and 5,681,847, which are here incorporated by reference in their entirety.

The invention provides in one of its aspects a pharmaceutical formulation comprising discodermolide, which hereinafter may be referred to as a formulation of the present invention.

In a preferred embodiment, the invention provides a pharmaceutical formulation in the form of an infusion concentrate which comprises discodermolide and a pharmaceutically acceptable organic solvent. The infusion concentration is diluted with a diluent vehicle to form a solution for infusion.

The first aspect of the present invention is an infusion concentrate comprising discodermolide and a pharmaceutically acceptable organic solvent chosen from any organic solvent known in the art. Said solvent may be used individually or in combinations with other solvents. Preferably the solvent is an alcohol with a carbon chain length of at least 2, e.g., C₂-C₅, e.g., C₂ or C₃ or C₄. Typical examples of alcohols are glycols, e.g., any glycol obtainable from an oxide, such as ethylene oxide, e.g., propylene glycol. Other examples are polyols, e.g., a polyalkylene glycol, e.g., poly(C₂-C₃)alkylene glycol or, e.g., polyethylene glycol. Other alcohols include absolute ethanol or glycerol. Most preferred is propylene glycol.

The discodermolide may be present with the organic solvent in an infusion concentrate in an amount of 0.1-50 mg/mL, e.g., 1-50 mg/mL or 0.5-50 mg/mL, more preferably 0.1-20 mg/mL, or 0.3-5 mg/mL or 0.5-4 mg/mL, 0.6-3 mg/mL, or 2 mg/mL.

A pharmaceutical formulation of the present invention in the form of an infusion concentrate may be produced by working up, e.g., dissolving discodermolide in a pharmaceutically acceptable solvent, optionally with other excipients. Preferably, no other excipients are present. However if other excipients are present, they are present preferably in an amount of <5%, e.g., <2%, e.g., between 0.1-1.5%.

Infusion concentrates of the present invention are conveniently stored in suitable containers, e.g., vials, double-chamber vial systems or ampoules. Typically the vials or ampoules are made from glass, e.g., borosilicate or soda-lime glass. The vials or ampoules may be of any volume conventional in the art, preferably they are of a size sufficient to accommodate 1-5 mL, more preferably 2 mL, of an infusion concentrate. The containers may accommodate preferably a stopper that can be pierced, e.g., a sterile rubber stopper, which may provide an appropriate hermetic seal with the container to allow for transfer of a liquid from or to the container.

The infusion concentrates of the present invention may be stable for an extended period of time, e.g., up to 12-36 months, e.g., 24 months at temperatures of at least 2-8° C. or at 25° C., as indicated in standard stability tests, e.g., as described in the examples.

In general, infusion concentrates are typically diluted to form an infusion solution before the compound is administered parenternally. It is understood that parenteral administration includes administration by infusion or injection.

Infusion concentrates of the present invention are not stable in aqueous media and therefore cannot be diluted in an aqueous medium, such as saline as is typical in the art, prior to i.v. administration. For example, in normal saline (0.9% sodium chloride), precipitation of the discodermolide is observed and the compound degrades to the hydrolysis product. Accordingly, it has been found that if the diluent vehicle comprises an organic solvent in saline, stability and solubility are maintained.

Accordingly, the present invention provides in another of its aspects a diluent vehicle comprising an organic solvent and saline. Examples of the organic solvent include propylene glycol, ethanol, Tween 80, benzoic acid, benzyl alcohol and mixtures thereof. Examples of diluent vehicles include ethanol in normal saline; ethanol and Tween 80 in normal saline; ethanol/benzoic acid in normal saline; and ethanol/benzoic acid/benzyl alcohol in normal saline. The ratio of organic solvent to saline will be such to obtain suitable solubility of discodermolide, however the amount of organic solvent is limited by practical limitations. If the ratio of organic solvent is too low, discodermolide is not soluble. However, the amount of organic solvent cannot be so high that too much organic solvent is administered to a patient. Preferred is 10-20% w/v ethanol in saline or 15-17% w/v ethanol in saline. The most preferred diluent vehicle comprises 16.3% w/v ethanol in saline, where each mL contains 7.3 mg NaCl and 163 mg ethanol.

The infusion concentrate and diluent vehicle are prepared and stored separately. Prior to administration the infusion concentrate and diluent vehicle are combined to form an infusion solution. The infusion solution so formed may be preferably used immediately or within a short time of being formed, e.g., within 8 hours. Alternatively, the infusion concentrate and a predetermined amount of diluent, may be loaded each into separate chambers of a double-chamber vial system and only mixed immediately prior to i.v. administration to a patient.

The amount of diluent used in admixture with the infusion concentrate to form an infusion solution may be chosen so as to obtain a desired concentration of discodermolide in the infusion solution. The amount of diluent used is also chosen so that the solution is stable long enough to be administered. Concentration of infusion solutions are 0.1-2.0 mg/mL or 0.1-1.5 mg/mL or preferably 0.5-8.0 mg/mL or 0.77 mg/mL. Such solutions are found to be stable for up to 8 hours.

Preferably the infusion solution of the present invention is prepared by mixing the infusion concentrate with a diluent, such as 16.3% w/v ethanol in saline in a suitable container. A preferred formulation comprises 1 mL of 2 mg/mL infusion concentrate diluted with 1.6 mL of 16.3% w/v ethanol in saline to yield an infusion solution containing 0.77 mg/mL discodermolide in 40% propylene glycol, 10% ethanol and 50% normal saline. This solution is administered via i.v. infusion, or push into Y tubing containing a 0.9% normal saline drip, directly into the vein and the drug solution residue in the tubing will be flushed with normal saline. The volume of discodermolide solution given via i.v. push will vary from 0.3 mL (at the starting does of 0.2 mg or 0.13 mg/m²) to 26 mL (for a dose of 20 mg or 11.43 mg/m²) or 43 mL (for a dose of 33 mg or 19.2 mg/m²). It has been found that direct i.v. infusion into the vein followed by a normal saline flush will not cause any precipitation of discodermolide.

Preferably the concentration of the infusion solution and dosage strength may be such to achieve an effective dose level of about 0.5-70 mg every 3 weeks, more preferably 1-30 mg every 3 weeks, or more preferably 30-40 mg every 3 weeks. The dose received by i.v. administration and the blood concentration may be determined accurately on the basis of known in vivo and in vitro techniques.

Infusion solutions according to the invention may comprise other excipients commonly employed in formulations to be administered by i.v. Excipients include antioxidants which may be chosen from any of those antioxidants known in the art and suitable for i.v. formulations. The amount of antioxidant may be determined by routine experimentation. As an alternative to the addition of an antioxidant, or in addition thereto, the antioxidant effect may be achieved by displacing oxygen (air) from contact with the infusion solution. This may be conveniently carried out by purging the container holding said infusion solution with an inert gas, e.g., nitrogen. Other excipients that may be present include isotonic agent or agents.

A pharmaceutical formulation of the present invention in suitable form for parenteral, e.g., i.v. administration, e.g., an infusion solution prepared by diluting an infusion concentrate with a diluent, may be filled in containers chosen from any conventional container which is non-reactive to said pharmaceutical formulations.

An infusion solution of the present invention is useful for treatment and prevention of malignant proliferative disorders, for example the indications and conditions disclosed in U.S. Pat. Nos. 5,010,099; 4,939,168; 5,840,75; and 5,681,847, the contents of which are incorporated herein by reference in their entirety. More specifically, they may be useful for the treatment of a tumor disease, e.g., a melanoma, ovarian cancer, pancreas cancer, neuroblastoma, head and neck cancer, bladder cancer, renal, brain, gastric or preferably a colorectal, prostate, breast, lung (especially non-small cell lung) or epithelial, especially epidermoid, e.g., cervical cancer. Formulations of the present invention are especially useful for solid tumors including tumors refractory to taxanes or epothiolones. Moreover, an infusion solution of the present invention is beneficial in treating conditions for which, and in the same manner as, Paclitaxel® is used. For certain tumors, discodermolides offer enhanced beneficial effects compared with Paclitaxel®.

Generally, the infusion solution of the present invention may be administered in an amount which is therapeutically effective against a proliferative disease which can be treated by administration of a discodermolide. Such proliferative diseases include any proliferative disease mentioned above, especially a tumor disease, the response to a therapeutically effective amount preferably manifesting itself in a diminished proliferation, e.g., diminished tumor growth, even more preferably tumor regression, or most preferably tumor disappearance. The exact amount and the duration of administration may depend upon the nature of discodermolide, the particular type of malignantly proliferating cells characteristic of the particular tumor, the seriousness of the condition, the rate of administration, as well as the patient's health and response to treatment.

Also, a pharmaceutical formulation of the present invention in suitable form for parenteral administration, e.g., an infusion solution prepared by diluting an infusion concentrate with a diluent vehicle, may be administered as a singe agent or combined with other tumor treatments known to a skilled person such as radiation, or administered as part of a combination therapy comprising at least one other chemotherapeutic agent. The administration of a combination of active agents may be simultaneous or consecutive, with either one of the active agents being administered first. The dosage of the active agents of a combination treatment may depend on effectiveness and site of action of each active agent, as well as synergistic effects between the agents used for combination therapy. It is understood that, parenteral administration includes administration by infusion or injection.

Other chemotherapeutic agents may include any chemotherapeutic agent that is or can be used in the treatment of tumor diseases, such as chemotherapeutics derived from the following classes:
1) alkylating agents, preferably cross-linking chemotherapeutics, preferably bis-alkylating agents;
2) anti-tumor antibiotics, preferably doxorubicin (Adriamycin®, Rubex®);
3) antimetabolites;
4) plant alkaloids;
5) hormonal agents and antagonists;
6) biological response modifiers, preferably lymphokines or interferons;
7) inhibitors of protein tyrosine kinases and/or serine/threonine kinases;
8) antisense oligonucleotides or oligonucleotide derivatives; or
9) miscellaneous agents or agents with other or unknown mechanism of action, preferably of the Taxane class, especially Taxotere® or most especially paclitaxel (Taxol®).

A diluted formulation of the present invention may, therefore, be useful as a single anti-cancer formulation or as part of a combination regimen for the treatment of various tumors.

The utility of infusion solutions of the present invention may be observed in standard clinical trials in, e.g., known indications of discodermolide dosages giving equivalent blood levels of discodermolide, e.g., using dosages in the range of about 0.1-75 mg/m² of discodermolide, preferably 0.5-60 mg/m² or 0.6-20 mg/m² for daily, weekly, every 2 weeks or every 3 weeks administration for a 75 kg mammal, e.g., an adult human of 1.73 m², and in standard animal models. Preferably, the dosage is from about 0.1-30 mg/m². Examples of preferred doses include 0.6 mg/m², 1.2 mg/m², 2.4 mg/m², 4.8 mg/m², 9.6 mg/m², 14.4 mg/m² and 19.2 mg/m². The preferred dose is 1-40 mg or 30-40 mg once every 3 weeks. This dose is administered to a human by i.v. administration.

The increased bioavailability of discodermolide administered in the form of a diluted formulation of the present invention, may be observed in standard animal tests and in clinical trials as described above. Naturally, the exact amounts of discodermolide and of the pharmaceutical formulation to be administered may depend on a number of factors, such as the condition to be treated, the exact discodermolide compound, the desired duration of treatment and the rate of administration of discodermolide. For example, the amount of discodermolide required and the administration rate thereof may be determined on the basis of known in vivo and in vitro techniques, e.g., as described above, determining how long a particular discodermolide concentration in the blood plasma remains at an acceptable level for a therapeutic effect.

In yet another aspect the invention provides a method of administering discodermolide to a subject in need of discodermolide treatment which comprises administering parenterally an infusion solution of the present invention to a subject in need of such treatment. More specifically, such a method of administering a discodermolide comprises:
(a) diluting a pharmaceutical formulation according to the invention, e.g., in the form of an infusion concentrate, with the diluent vehicle, to form a solution suitable for parenteral, e.g., i.v. administration; and
(b) administering such infusion solution to the subject.

In yet another aspect the invention provides use of discodermolide in the manufacture of a medicament suitable for parenteral administration.

The invention is illustrated by way of the following examples which are not intended to limit the scope of the present invention.

### EXAMPLE 1

An infusion concentrate comprising 2 mg of discodermolide is dissolved in 98-100% propylene glycol (PG) (1.0 mL) and charged to 6 mL vials. The vials are used for storage and shipment. The vials are stable for a period of at least 2 years at a temperature of 2-8° C. The vehicle is prepared in a separate 2 mL single dose vial. Each mL of solution contains 7.3 mg of sodium chloride USP, and 163 mg of ethanol (EtOH) in water for injection(saline) (16.3% ethanol in 0.9% sodium chloride solution, 2 mL fill). The storage condition is 25° C. for the vehicle. Prior to i.v. administration, 1.6 mL of the vehicle is withdrawn with a sterile disposable syringe and injected into the vial containing the discodermolide. The solution is gently shaken until a clear homogeneous solution for infusion is obtained containing 0.77 mg/mL discodermolide in 40% propylene glycol, 10% ethanol and 50% normal saline. The ratio of discodermolide concentrate to the diluent vehicle in the diluted solution is 1:1.6 v/v. The i.v. solution thus formed is stable for a period of 8 hours at room temperature.

### EXAMPLES 2-10

The solubility of discodermolide in various solvent systems is measured by HPLC and the results shown below:

TABLE 1

| Example | Solvent system | Solubility [mg/mL] |
|---|---|---|
| 2 | 100% PG | >20 |
| 3 | 100% ethanol | >20 |
| 4 | 100% PEG 300 | >20 |
| 5 | 60% PG/40% normal saline | 0.53 |
| 6 | 20% PG/80% normal saline | 0.13 |
| 7 | 60% ethanol/40% normal saline | 2.3 |
| 8 | 30% ethanol/70% normal saline | 0.2 |
| 9 | 60% PEG 300/40% normal saline | 0.4 |
| 10 | 20% PEG 300/80% normal saline | 0.02 |

### EXAMPLE 11

Three discodermolide formulations are prepared and evaluated in an in vitro precipitation study to predict the precipitation potential of discodermolide in the formulations upon injection. The formulations studied are:

Formulation A: 2 mg/mL discodermolide in 8.3% EtOH/16.7% cremophor EL in D5W

Formulation B: 2 mg/mL discodermolide in 60% PG/40% normal saline

Formulation C: 2 mg/mL of discodermolide in 50% PG/10% EtOH/40% normal saline

Under static conditions, precipitation of discodermolide in Formulation A occurred at very low drug concentrations, i.e., 0.05 mg/mL, in 66.7 mM isotonic phosphate buffered saline (ISPB)/albumin, whereas the two propylene glycol-based formulations did not precipitate under similar conditions. The results suggest that the potential for discodermolide to precipitate upon i.v. administration is low in propylene glycol formulations but high in cremophor EL/EtOH/D5W formulations.

### EXAMPLE 12

The stability of propylene glycol infusion concentrates comprising discodermolide at different concentrations and various temperatures is determined. Table 2 describes the amount of degradation product formed over a period of up to 3 months. The stability is analyzed by determining formation of degradation products in each of the infusion concentrates as a function of time and temperature. Each sample is analyzed by HPLC with a UV detector. No significant changes in the discodermolide content is observed after 3 months at 5° C.; the total impurity amounts are below the proposed specification limits of =2.0%. Under accelerated conditions, the levels of impurities increased at both 25° C./60% relative humidity (RH) and 45° C./60% RH.

TABLE 2

| | 0.6 mg/mL | | 2.0 mg/mL | | 3.0 mg/mL | |
|---|---|---|---|---|---|---|
| | Assay | Total | Assay | Total | Assay | Total |
| | (% label | Impurities | (% label | Impurities | (% label | Impurities |
| Storage Conditions | claim) | (%) | claim) | (%) | claim) | (%) |
| 0-time | 100.4 | 0.18 | 99.9 | 0.19 | 102.7 | 0.19 |
| 5° C. | | | | | | |
| 0.5 month | 103.2 | 0.16 | 103.2 | 0.16 | 101.5 | 0.16 |
| 1 month | 102.6 | 0.19 | 100.5 | 0.18 | 102.0 | 0.19 |
| 2 months | 103.3 | 0.27 | 102.5 | 0.18 | 104.1 | 0.17 |
| 3 months | 103.8 | 0.29 | 101.0 | 0.54 | 104.4 | 0.29 |
| 25° C./60% RH | | | | | | |
| 0.5 month | 102.8 | 0.65 | 101.1 | 0.55 | 102.4 | 0.35 |
| 1 month | 101.9 | 1.08 | 99.6 | 0.78 | 101.3 | 0.52 |
| 2 months | 102.5 | 1.18 | 101.4 | 1.18 | 103.1 | 0.69 |
| 3 months | 102.4 | 1.43 | 100.9 | 1.48 | 102.1 | 0.69 |
| 45° C./60% RH | | | | | | |
| 0.5 month | 102.0 | 1.49 | 100.3 | 1.17 | 100.9 | 0.58 |
| 1 month | 101.6 | 1.91 | 100.0 | 1.69 | 101.5 | 0.70 |

### EXAMPLE 13

The stability of an infusion concentrate of 16 mg/mL in propylene glycol at different temperatures is determined. Table 3 describes the amount of degradation product formed over a period of up to 12 weeks. The stability is analyzed by determining formation of degradation products in each of the infusion concentrates as a function of time and temperature. Each sample is analyzed by HPLC with a UV detector. The degradation profile and long term stability is comparable to 2.0 mg/mL as shown in Example 2.

TABLE 3

| | | 16 mg/mL |
|---|---|---|
| | Assay | Total |
| Storage Conditions | (% label claim) | Impurities (%) |
| 0-time | 103.9 | 0.59 |

5° C.

| | | |
|---|---|---|
| 4 weeks | 102.2 | 0.60 |
| 8 weeks | 101.4 | 0.48 |
| 12 weeks | 100.7 | 0.49 |

25° C./60% RH

| | | |
|---|---|---|
| 3 weeks | 100.7 | 0.56 |
| 4 weeks | 103.6 | 0.66 |
| 8 weeks | 100.5 | 0.68 |
| 12 weeks | 100.1 | 0.65 |

50° C./75% RH

| | | |
|---|---|---|
| 3 weeks | 99.4 | 0.65 |
| 4 weeks | 101.6 | 0.65 |
| 8 weeks | 99.5 | 0.65 |
| 12 weeks | 99.2 | 0.62 |

### EXAMPLE 14

In this example, stability of discodermolide infusion concentrates diluted 1:5 with 20/80 propylene glycol/0.9% sodium chloride solution under ambient conditions is measured. Table 4 describes the amount of degradation product formed over a period of up to 24 hours. The stability is analyzed by determining formation of degradation products in each of the infusion solutions as a function of time and temperature. Each sample is analyzed by HPLC with a UV detector. The solution for infusion was prepared by diluting discodermolide concentrates, 0.6 mg/mL, 2.0 mg/mL and 3.0 mg/mL, with 20/80 propylene glycol/0.9% sodium chloride solution in a 1:5 ratio. The discodermolide concentrate solutions of 0.6 mg/mL, 2.0 mg/mL and 3.0 mg/mL yielded discodermolide solutions for infusions 0.12 mg/mL, 0.4 mg/mL and 0.6 mg/mL, respectively. The stabilities of these solutions were evaluated over a 24-hour period under ambient conditions and the data presented in Table 4.

TABLE 4

| | | |
|---|---|---|
| Solution | | |
| for Infusion | Assay | Total Impurities |
| Time (hours) | (% label claim) | (% area) |

0.12 mg/mL

| | | |
|---|---|---|
| 0 | 105.6 | 0.17 |
| 2 | 105.6 | 0.17 |
| 4 | 106.4 | 0.21 |
| 8 | 107.0 | 0.25 |
| 24 | 104.6 | 0.33 |

0.4 mg/mL

| | | |
|---|---|---|
| 0 | 102.3 | 0.23 |
| 2 | 101.8 | 0.97 |
| 4 | 102.2 | 1.84 |
| 8 | 102.0 | 3.35 |
| 24 | 94.8 | 6.21 |

0.6 mg/mL

| | | |
|---|---|---|
| 0 | 103.0 | 0.28 |
| 2 | 100.0 | 1.49 |
| 4 | 99.4 | 2.19 |
| 8 | 98.8 | 3.68 |
| 24 | 89.9 | 8.90 |

### EXAMPLE 15

In this example, stability of discodermolide in solution diluted 1:1.6 with 16.3% w/v, ethanol in 0.9% sodium chloride solution (0.77 mg/mL) under ambient conditions is measured. Table 5 describes the amount of degradation product formed over a period of up to 24 hours. The stability is analyzed by determining formation of degradation products in each of the infusion solution as a function of time and temperature. Each sample is analyzed y HPLC with a UV detector. The solution for infusion was prepared by diluting a 2.0 mg/ml discodermolide concentrate with 16.3% ethanol in 0.9% sodium chloride solution in a 1:1.6 ration. The stabilities of this solution was evaluated over a 24-hour period under ambient conditions and the data presented in Table 5.

TABLE 5

| Time (hours) | Total impurities (% area) |
|---|---|
| 0 | 0.20 |
| 2 | 0.23 |
| 4 | 0.34 |
| 8 | 0.47 |
| 24 | 0.80 |

### EXAMPLE 16

Table 6 shows the effect of organic solvent ratio variation on the solubility of discodermolide.

TABLE 6

| | Deviation | | Deviation | | |
|---|---|---|---|---|---|
| PG | (%) from | EtOH | (%) from | Normal saline | Solubility |
| (%, ^{w}/ᵥ) | the target | (%, ^{w}/ᵥ) | the target | (%, ^{w}/ᵥ) | (mg/mL) |
| 40 | 0 | 10 | 0 | 50 | 0.96 |
| 40 | 0 | 9.6 | −4 | 50.4 | 0.87 |
| 40 | 0 | 9 | −10 | 51 | 0.77 |
| 40 | 0 | 8 | −20 | 52 | 0.62 |
| 38 | −5 | 9.5 | −5 | 52.5 | 0.70 |
| 38 | −5 | 9 | −10 | 53 | 0.64 |

### EXAMPLE 17

The solubility and stability of discodermolide in 40% propylene glycol/10% ethanol 50% normal saline diluted with hydrotropic agents is shown in Table 7.

TABLE 7

| | | Area |
|---|---|---|
| | Discodermolide | ratio of Acid/ |
| | Solubility | Discodermolide |
| Vehicle | (mg/mL) | (%) |
| 40% PG/10% EtOH in normal saline | 0.91 | 4.2 |
| 40% PG/10% EtOH/4% benzoic acid | 1.9 | 1.8 |
| in normal saline | | |
| 40% PG/10% EtOH/4% benzoic acid/ | 2.2 | 1.5 |
| 1% benzyl alcohol in normal saline | | |

We claim:

1. A pharmaceutical formulation in the form of an infusion concentrate comprising discodermolide and a pharmaceutically acceptable organic solvent selected from an alcohol.

2. A pharmaceutical formulation according to claim 1, wherein the pharmaceutically acceptable organic solvent is a propylene glycol.

3. A pharmaceutical formulation according to claim 1, wherein the discodermolide is at a concentration of 0.1-20 mg/mL.

4. A pharmaceutical formulation according to claim 1, wherein the discodermolide is at a concentration of 0.6-3 mg/mL.

5. A pharmaceutical formulation according to claim 1, wherein the discodermolide is at a concentration of 2 mg/mL.

6. An infusion solution comprising an infusion concentrate according to claim 1 and a diluent vehicle selected from a mixture of saline and pharmaceutically acceptable solvents and mixtures thereof.

7. An infusion solution according to claim 6, wherein the solvent is selected from propylene glycol, ethanol, benzoic acid, benzoate, benzyl alcohol and mixtures thereof.

8. An infusion solution according to claim 6, wherein the diluent vehicle is selected from ethanol in saline.

9. An infusion solution according to claim 6, wherein the diluent vehicle is 10-20% w/v ethanol in saline.

10. An infusion solution according to claim 6, wherein the diluent vehicle is 16.3% w/v ethanol in saline.

11. A pharmaceutical kit comprising an infusion concentrate of discodermolide in an organic solvent and a diluent vehicle.

12. A pharmaceutical kit comprising an infusion concentrate of discodermolide in an organic solvent and a diluent vehicle wherein the infusion concentrate and diluent vehicle are in separate containers.

13. A method of administering discodermolide to a subject in need of discodermolide treatment which comprises administering parenterally an infusion solution according to claim 6 to a subject in need of such treatment.

14. A method of administering a discodermolide for the treatment of a proliferative disease, sensitive to treatment with discodermolide, to a mammal in need of such treatment in a therapeutically effective amount which comprises:
(a) diluting an infusion concentrate according to claim 1 with a diluent vehicle to form an infusion solution; and
(b) administering the infusion solution by i.v. to the subject.

15. A method of administering a discodermolide for the treatment of a proliferative disease sensitive to treatment with discodermolide to a mammal in need of such treatment in a therapeutically effective amount which comprises:
(a) diluting an infusion concentrate comprising discodermolide with a diluent comprising 16.3% w/v ethanol in saline to form an infusion solution; and
(b) administering the infusion solution by i.v. to the subject.

16. A method of administering a discodermolide for the treatment of a proliferative disease sensitive to treatment with discodermolide to a mammal in need of such treatment in a therapeutically effective amount which comprises:
(a) diluting a 2 mg/mL infusion concentrate comprising discodermolide with a diluent comprising 16.3% w/v ethanol in saline in a 1:1.6 ratio to form an infusion solution; and
(b) administering the infusion solution by i.v. to the subject.

17. A method according to claim 14, wherein step (a) is completed 8 hours or less prior to administration.

18. A method of preparing a infusion solution for administration comprising:
(a) preparing a solution of discodermolide in an organic solvent; and
(b) diluting the solution of step (a) with a vehicle comprising saline and ethanol,
wherein step (b) is done 8 hours or less prior to administration.
